(19) United States
(12) Patent Application Publication
(10) Pub. No.: US 2006/0105061 A1
(43) Pub. Date: May 18, 2006

(54) Galenical composition

(76) Inventors: Shinhan Sha, Tokyo (JP); Francesco Marotta, Milano (IT)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/240,474

(22) Filed: Oct. 3, 2005

(30) Foreign Application Priority Data
Oct. 22, 2004 (JP) 2004-307676

(51) Int. Cl. A61K 36/258; A61K 36/484

(52) U.S. Cl. 424/728; 424/773; 424/757

(57) ABSTRACT

A method for preventing metal-induced oxidative damage of hepatocytes, for inhibiting hepatocellular carcinogenesis, or for increasing carcinogen-metabolizing enzyme activity, which comprises administering to a subject an effective amount of an extract of a galenical composition comprising Panax pseudoginseng, Eucommiae ulmoides and Polygonati Rhizoma as essential components.

### BACKGROUND OF THE INVENTION

(1) Field of the Invention

The present invention relates to a novel application of a galenical composition (crude drug composition, herbal drug composition) comprising “Denhichi” (Panax pseudoginseng), “Tochu” (Eucommiae ulmoides (Eucommia Bark)) and “Osei” (Polygonati Rhizoma) as essential components. More particularly, the present invention relates to a use of the aforementioned galenical composition (crude drug composition, herbal drug composition) for preventing metal-induced damage of liver, for inhibiting neoplastic liver disorder, and for preventing induction of tumor.

(2) Description of the Related Art

Iron performs an important role in cellular and organismal physiology because it is a cofactor for binding to various proteins essential for cell function. However, iron may accumulate in biological system and, in particular, in the liver for various pathological factors such as due to a consequence of genetic defects in the gut absorption or following repeated parenteral administration of nutrients. It is known that abundant free iron acts as a strong hepatotoxin as well as pro-fibrogenetic factor especially in the presence of chronic alcohol consumption, viral hepatitis or hepatotoxic xenobiotics. In the presence of an excess amount of a metal catalyst such as iron, oxidative stress is a common finding since iron becomes a souce of a number of free radical species while also being an inducer of lipid peroxidation. Similarly, copper represents another strong cause of oxidative stress as it occurs in copper-storage diseases such as Wilson's disease. Under physiological and pathological processes, the homeostasis of free radical balance is maintained by a complicated system where endogenous and exogenous antioxidant protecting cells and tissues interplay with the generation of reactive oxygen species which may bring about damaging effects. In particular, there is clear evidence that the presence of an excess amount of iron in liver is associated with hepatocellular injury, activation of inflammatory cascade, fibrosis and also hepatocellular carcinoma (Non-Patent Document 1). It has been suggested that either copper and iron, both important transition metals in the body, may participate in the induction of DNA damage and oncogenesis, being mutagenic in bacteria and in Chinese hamster (transgenic strain) lung cells (Non-Patent Document 2). Overall, on the clinical ground it has been shown that a direct correlation between increased body iron stores and an increased risk of cancer of all organs and tissues in individuals even not suffering from iron overload diseases. This is not surprising when considering that catalysis caused by iron or copper leads to the generation of reactive oxygen species that can avidly attack biomolecules, with the consequent lipid peroxidation of cellular membrane, protein oxidation and DNA damage which involves site-specific Fenton-type chemistry. We have previously shown either in vitro and in vivo experimental studies that “Youjo-Hensikoh” (hereinafter also called as YHK) exerts a potent protective effect against hepatocellular damage and on liver microcirculation in an ischemia-reperfusion model (Non-Patent Documents 3 and 4).

Nowadays there is a concern over the possible involvement of xenobiotics in carcinogenesis and this has led to a solid research stream in both humans and rodents. Indeed, DNA can be damaged along the whole process of absorption of carcinogens into the body, distribution to most sensitive tissues, metabolism which gives rise to a further form reacting with DNA, detoxification, and excretion. There are many genotoxic carcinogens occurring naturally in the environment surrounding man, including the large group of heterocyclic amine mutagens. For instance, 2-amino-3,8-dimethylimidazo[4,5-f]quinoxaline (MeIQx), one of food-derived agent, might develop an overt hepatocellular carcinomas with treatment at high doses, and induce in rats DNA adduct formation in the liver. In clinical practice, it has been shown that the human daily intake of MeIQx is estimated to be 0.2 to 2.6 μg/subject and this substance has been recovered and quantified in the urine of healthy volunteers after eating cooked meat. However, more important, MeIQx-DNA adducts have been found in kidney and colon tissues in man (Non-Patent Document 5). Besides the importance of the detection and removal of such carcinogenetic agents, a protective dietary approach would represent an ideal countermeasure, given the overwhelming constant exposure to xenobiotics. Several natural compounds have been proposed while only few have proven to possess a validated property in experimental tests. We have experimentally shown an effect on hepatocellular damage and liver microcirculation in an ischemia-reperfusion model. Indeed, on the clinical ground, to the contrary of many galenicals experimentally tested, the present composition YHK has shown to significantly lower within two-three weeks the ALT level in the majority of HCV-related liver disease patients (Non-Patent Document 6) and, moreover, to decrease Maruyama score in an awarded pilot clinical study done on the same subjects (Non-Patent Document 7). The rat liver represent an ideal model to study the whole sequence of cancer initiation and development since within a few days after administration of various toxic hepatocarcinogens from xenobiotics single hepatocytes express placental glutathione S-transferase (single placental glutathione S-transferase-positive (GST-P) cells). A separate population of such GST-P single cells develops into GST-P foci, which increase further in number and size on treatment with tumor promoters to a final transfomation into GST-P tumors. Therefore, the number and size of GST-P foci can be used as quantitative indicators of subsequent cancer risk although not all positive liver foci may necessarily develop tumors. There is an over 90% correlation between the two events while the assay also correlates with the incidence of hepatocellular carcinomas in parallel long-term studies.

Hepatocellular carcinoma (HCC) is one of the most frequent cancers worldwide and is most often associated with exposure to environmental factors such as aflatoxin B1, hepatitis viruses B and C, and alcohol consumption. Agents with tumor promoting activity in the liver generally cause enzyme induction, enlargement of the liver by hypertophy and/or hyperplasia, an increase in DNA synthesis and/or decrease in apoptotic activity, which is more pronounced in preneoplastic than in unaltered cells, and preferential growth stimulation of precancerous lesions. Fruits, vegetables, vitamins and several herbs with diversified pharmacological properties have been shown to be a rich source of cancer chemopreventive agents. Though these agents can be targeted for treatment at either initiation, promotion or progression stages of multistep processes of carcinogensis. Many of these actions have been related to the abilities to enhance the activities of carcinogen metabolizing enzymes and by binding with toxicants thus reducing their effective critical concentrations. Hepatic drug metabolizing system consists of mixed-function oxidase or monooxygenase enzymes including phase I enzymes such as cytochrome P450, cytochrome b5 and NADPH-cytochrome P450 reductase and phase II enzymes such as glutathione-S-transferase (GST), sulfatase and UDP-glucuronyl transferase. Chemopreventive agents acts as antioxidant and counteract the increase of oxidants generated by toxicants. Among phase I enzymes, CYP 1A1 is primarily involved in the metabolism of polycyclic aromatic hydrocarbons, whereas CYP 1A2 preferentially metabolizes heterocyclic amines and aflatoxin B1. These isoforms may play a very important role in activation of these environmental carcinogens. CYP 1A2 is normally expressed in liver. The level of CYP 1A1 expression in the liver is substantially lower than for CYP 1A2 and inducers of CYP 1A may have considerable potential ability for toxicity, carcinogenicity. GST belongs to a superfamily of multifunctional isoenzymes categorized into three major classes, α, μ and π. It has been suggested that the GST α possesses high catalytic efficiency towards aflatoxin B₁-8,9-epoxide, the reactive intermediate of aflatoxin B₁, the fungus mycotoxin while the GST μ isoenzyme is most efficient in forming a conjugation of glutathione with carcinogen 4-nitroquinoline-1-oxide (Aceto) and that GST π metabolites preferentially conjugate 7β, 8α-dihydroxy-9α, 10α-oxy-7,8,10-tetrahydrobenzo(a)pyrene, the ultimate carcinogenic metabolite of benzo(a)pyrene including aflatoxin B₁ or 4-nitroquinoline-1-oxide. Phase I enzymes, which include cytochrome P450, can metabolize not only lipophilic compounds to more polar products but, under some circumstances, can lead to generation of highly reactive electrophiles. Therefore, the balance between phase II and phase I enzymes is likely to be important for determining cellular sensitivity to environmental chemicals.

[Patent Document 1] U.S. Pat. No. 6,586,017

[Patent Document 2] JP-A-2000-139405

[Patent Document 3] JP-A-11-289995

[Non-Patent Document 1] Pietrangelo A, J Hepatol 1998; 28(suppl 1):8-13

[Non-Patent Document 2] Ma Y et al., Pathol Int. April 1997;47(4):203-8

[Non-Patent Document 3] Marotta F, Rouge A, Harada M, Anzulovic H, Ideo G M, Yanaihara N, Princess G, Ideo G, Biomed Res 2001; 22:167-174

[Non-Patent Document 4] Marotta F, Bertuccelli J, Albergati F, Harada M, Safran P, Yanaihara N, Ideo G, Biomed Res 2001; 22:221-227

[Non-Patent Document 5] Totsuka, Y et al., Carcinogenesis 1996; 17:1029-1034

[Non-Patent Document 6] Sha S, Harada M, Yanaihara N, IASL-APASL Joint meeting 2000, New Insights of Hepatology in the 21st century. Jun. 2-7, 2000 Fukuoka, Japan

[Non-Patent Document 7] Harada M, Marotta F, Sha S H, Minelli E. Y H K, First JSH Single Topic Conference “Therapy of viral hepatitis and prevention of hepatocellular carcinoma” Nov. 14-15. 2002, Yamanashi, Japan

### SUMMARY OF THE INVENTION

Thus, it is an object of the present invention to provide a composition having a protective effect against hepatocytes oxidative damage by metals such as iron, copper and vanadium which is also known to trigger oxidative damage to cellular membranes and nuclear DNA. In the present invention, the protective effect of YHK against metal-induced oxidative damage of hepatocytes was examined in vitro.

It is another object of the present invention to provide a composition having a protective effect in the early stage of chemical hepatocellular carcinogenesis.

It is a further object of the present invention to provide a composition having a protective effect against these enzymes and liver antioxidant, because bioactivation of precarcinorgens and detoxification of ultimate carcinogens are mainly carried out by drug metabolizing enzymes in the liver and these may be influenced by specific nutrients.

The present invention is as follows:
(1) A galenical composition (crude drug composition, herbal drug composition) comprising Panax pseudoginseng, Eucommiae ulmoides and Polygonati Rhizoma as essential components, for preventing metal-induced oxidative damage of hepatocytes, for inhibiting hepatocellular carcinogenesis, or for increasing carcinogen-metabolizing enzyme activity.
(2) The galenical composition (crude drug composition, herbal drug composition) according to the aforementioned (1), further comprising at least one selected from the group consisting of Licorice root, Panax ginseng and honey.
(3) The galenical composition (crude drug composition, herbal drug composition) according to the aforementioned (1), further comprising Licorice root and Panax ginseng.
(4) The galenical composition (crude drug composition, herbal drug composition) according to any one of the aforementioned (1) to (3), wherein the composition is for preventing metal-induced oxidative damage of hepatocytes, and the metal is at least one selected from the group consisting of iron, copper and vanadium.
(5) The galenical composition (crude drug composition, herbal drug composition) according to the aforementioned (4), wherein the metal is iron.
(6) The galenical composition (crude drug composition, herbal drug composition) according to the aforementioned (4) or (5), wherein the metal-induced oxidative damage of hepatocytes is lipid peroxidation and/or lysosome alteration.
(7) The galenical composition (crude drug composition, herbal drug composition) according to any one of the aforementioned (1) to (3), wherein the composition is for inhibiting hepatocellular carcinogenesis.
(8) The galenical composition (crude drug composition, herbal drug composition) according to any one of the aforementioned (1) to (3), wherein the composition is for increasing carcinogen-metabolizing enzyme activity.
(9) The galenical composition (crude drug composition, herbal drug composition) according to the aforementioned (8), wherein the carcinogen-metabolizing enzyme is a cytochrome P450 (CYP) isoform, phase I enzyme and phase II enzyme.
(10) The galenical composition (crude drug composition, herbal drug composition) according to the aforementioned (8), wherein the carcinogen-metabolizing enzyme is glutathione peroxidase, glutathione reductase, glucose-6-phosphate dehydrogenase, catalase, glutathione S-transferase or quinone reductase.
(11) A method for preventing metal-induced oxidative damage of hepatocytes, for inhibiting hepatocellular carcinogenesis, or for increasing carcinogen-metabolizing enzyme activity, which comprises administering to a subject an effective amount of an extract of a galenical composition (crude drug composition, herbal drug composition) comprising Panax pseudoginseng, Eucommiae ulmoides and Polygonati Rhizoma as essential components.
(12) The method according to the aforementioned (11), wherein the galenical composition (crude drug composition, herbal drug composition) further comprises at least one selected from the group consisting of Licorice root, Panax ginseng and honey.
(13) The method according to the aforementioned (11), wherein the galenical composition (crude drug composition, herbal drug composition) further comprises Licorice root and Panax ginseng.
(14) The method according to any one of the aforementioned (11) to (13), wherein the method is for preventing metal-induced oxidative damage of hepatocytes, and the metal is at least one selected from the group consisting of iron, copper and vanadium.
(15) The method according to the aforementioned (14), wherein the metal is iron.
(16) The method according to the aforementioned (14) or (15), wherein the metal-induced oxidative damage of hepatocytes is lipid peroxidation and/or lysosome alteration.
(17) The method according to any one of the aforementioned (11) to (13), wherein the method is for inhibiting hepatocellular carcinogenesis.
(18) The method according to any one of the aforementioned (11) to (13), wherein the method is for increasing carcinogen-metabolizing enzyme activity.
(19) The method according to the aforementioned (18), wherein the carcinogen-metabolizing enzyme is a cytochrome P450 (CYP) isoform, phase I enzyme and phase II enzyme.
(20) The method according to the aforementioned (18), wherein the carcinogen-metabolizing enzyme is glutathione peroxidase, glutathione reductase, glucose-6-phosphate dehydrogenase, catalase, glutathione S-transferase or quinone reductase.

The galenical composition (crude drug composition, herbal drug composition) of the present invention can be used for preventing metal-induced damage of liver, for inhibiting neoplastic hepatic damage, and for preventing tumor induction.

### BRIEF DESCRIPTION OF THE DRAWINGS

FIG. 1 is a graph showing the results of lysosomal fragility test, and shows the effects of YHK and sylibin on the LHD release when cultured hepatocytes are challenged by metal ions.

FIG. 2 is a graph showing the results of lysosome fragility test, and shows the effects of YHK and sylibin on the β-galactosidase release induced by metal ions in lysosomal fractions.

FIG. 3 is a graph showing DPPH radical-scavenging activity of YHK and sylibin in lysosomal fractions.

### DETAILED DESCRIPTION OF THE INVENTION

The galenical composition of the present invention comprises Panax pseudoginseng, Eucommiae ulmoides and Polygonati Rhizoma as essential components. Preferably, the galenical composition of the present invention further comprises at least one selected from the group consisting of Licorice root, Panax ginseng and honey, and more preferably, comprises Licorice root and Panax ginseng.

As such a galenical composition, “Youjo-Hensikoh” (hereinafter also called as YHK) is known (available from Kyotsujigyo Inc. (Tokyo)). YHK is a galenical composition which comprises, if necessary, Licorice root and/or honey as well as Panax pseudoginseng, Eucommiae ulmoides and Polygonati Rhizoma. A galenical composition comprising Panax pseudoginseng, Eucommiae ulmoides and Polygonati Rhizoma, and also YHK are described in U.S. Pat. No. 6,586,017 (July 2003) (Patent Document 1).

Panax pseudoginseng (“Denhichi”) is also called as “Sanshichi-ninjin”, which uses the root of Panax pseudo-ginseng Wall produced in China. This galenical is known to ameliorate a disorder of lipid metabolism and inhibit elevated blood pressure and pain.

In the galenical composition of the present invention, Panax pseudoginseng is used in an amount of 10 to 90% by weight, preferably 30 to 90% by weight.

Eucommiae ulmoides (or Eucommia Bark, “Tochu”) is generally comprised of dried bark of a deciduous arbor, Eucommiae ulmoides of the family Eucommiaceae. This galenical is known to lower elevated blood pressure and high blood lipid level. In the present invention, Eucommiae ulmoides refers to be from a dry substance of not only bark, but also leaflets, fruit and/or wood.

In the galenical composition of the present invention, Eucommiae ulmoides is used in an amount of 10 to 90% by weight, preferably 10 to 70% by weight or 40 to 90% by weight.

“Denhichi-Tochu-Sei” is known as a galenical composition comprising Panax pseudoginseng and Eucommiae ulmoides. This composition was developed firstly by us, and obtained by extracting with hot water a galenical mixture containing Panax pseudoginseng and Eucommiae ulmoides as the main components (JP-A-2000-139405 (Patent Document 2) and JP-A-11-289995 (Patent Document 3)). “Denhichi-Tochu-Sei” preferably contains Panax ginseng and honey, in addition to the main components, Panax pseudoginseng and Eucommiae ulmoides.

Polygonati Rhizoma is also called as “Osei” or Siberian Solomonseal Rhizome, which is a dried rhizome of an origin plant belonging to the genus Polygonatum. Polygonati Rhizoma is a traditional galenical useful for improvement of nutritional state and health promotion.

In the galenical composition of the present invention, Polygonati Rhizoma is used in an amount of 20% by weight or less, preferably 4 to 20% by weight, more preferably 6 to 12% by weight.

As origin plants for Polygonati Rhizoma, mention may be made of Polygonatum falcatum A. Gray, Polygonatum multiflorum, Polygonatum odoratum, Polygonatum odoratum (Mill.) Druce, Polygonatum cyrtonema Hua, Polygonatum sibiricum Redoute, Polygonatum sibiricum Delar. ex Redoute, Polygonatum kingianum Coll. et Hemsl., Polygonatum stenophyllum Maxim., Polygonatum involucratum Maxim., Polygonatum macropodium Turez., Polygonatum cirrhifolium (Wall.) Royle, Polygonatum prattii Baker, Polygonatum punctatum Royle ex Kunth, Polygonatum zanlanscianense Pamp., Polygonatum curvistylum Hua, Polygonatum tessellatum Wang et Tang, Polygonatum roseum (Ledeb.) Kunth, P. verticillatum (L.) All., P. curvistylum Hua, P. erythrocarpum Hua, P. filipes Merr., P. lasianthum Maxim, and the like.

It is known that Polygonati Rhizoma is effective for ameliorating the symptom or liver function of a patient suffering from chronic hepatitis, and further, the alcohol extract of Polygonati Rhizoma shows a preventive effect on carbon tetrachloride-induced hepatopathy of mice. Polygonati Rhizoma has an antiviral effect on hepatitis B virus or the like, but it also has problems that discontinuation of the medication leads to the return to the first, and further, particularly in the case of Japanese people, the medication with Polygonati Rhizoma alone causes large gastric discomfort, and Polygonati Rhizoma is poorly absorbed in a human body.

Licorice root (“Kanzo”) is a dried root and a dried stolon of Glycyrrhiza glabra Linn, a plant belonging to the genus Glycyrrhiza of the family Leguminosae, and also called as “glycyrrhiza”, “licorice”, “liquorice”, “glycyrrhiza radix” or the like.

In the galenical composition of the present invention, Licorice root is used in an amount of 0 to 15% by weight, preferably 4 to 15% by weight, more preferably 6 to 11% by weight.

A pharmaceutical preparation comprising glycyrrhizin, the main component of Licorice root is recognized to increase the level of serum transaminase and ameliorate various hepatic damage-associated symptoms by a double blind experiment. Glycyrrhizin is recognized to have protective effects on a hepatocyte membrane and a liver, such as inhibition of experimental hepatitis caused by carbon tetrachloride or inhibition of hepatocellular damage (Y. Ishii Japan J. pharmaco, 120, 71 (1971), Susumu Okabe: Oyo Yakuri, 7, 87 (1973)). As described above, Licorice root has antiviral effects against hepatitis B virus or the like, although the effects are smaller than those of Polygonati Rhizoma. However, Licorice root also has the same problems as in Polygonati Rhizoma as described above.

In the galenical composition of the present invention, Panax ginseng is used in an amount of 0 to 20% by weight, preferably 5 to 20% by weight.

In the galenical composition of the present invention, honey is used in an amount of 0 to 30% by weight.

The galenical composition of the present invention shows excellent concerted effects that the composition is easily absorbable in a human body, the medication effects appear in a short period of time, and the effects continue even after stopping the medication.

Next, a method for preparing the galenical composition of the present invention will be described below.

The extracts of Panax pseudoginseng and Eucommiae ulmoides are obtained, for example, by chipping dried Eucommiae ulmoides (bark, leaflets, fruit, wood) and dried Panax pseudoginseng, and then extracting them with hot water of 60° C. to 100° C. or with ethanol or a mixed solution of ethanol and water (100:0 to 0:100) of room temperature to 100° C. for 0.5 to 2 hours (extraction step) followed by each step of filtration, purification and concentration.

The extract of Polygonati Rhizoma is also obtained by chipping dried Polygonati Rhizoma, extracting it with hot water and concentrating the resulting liquid. Powdered Polygonati Rhizoma is obtained by drying the extract of Polygonati Rhizoma with hot air.

To both of the resulting extracts, Licorice root extract, Panax ginseng extract, honey or the like are added as an optional component(s), and then they are mixed together (mixing step). The galenical composition of the present invention in a powder or powdered granule form can be obtained by drying the mixture with hot air (drying step). A tablet can be obtained by adding an emulsifier to the resulting mixture, molding the mixture followed by drying with hot air. In this manner, the galenical composition of the present invention can be obtained as powder, granule, tablets or liquid (drink).

Honey, vegetable oil, xylitol or the like is used as an emulsifier. The weight ratio of the emulsifier is desirably 0 to 20% by weight.

The tablet obtained by drying the above described mixed powder of galenical component extracts and emulsifier with hot air is suitably 0.1 to 0.5 g/tablet, especially 0.25 g/tablet.

In humans, the dose of the galenical composition of the present invention is about 0.1 to 2 g/kg per day, preferably 0.1 to 0.25 g/kg per day.

The invention will be further described by the Examples in the following, but the present invention will not be limited in any extent by the Examples.

Hepatocytes are oxidatively damaged by metal ions, and leak (release) lactate dehydrogenase (LDH) and β-galactosidase. Lipid peroxidation and/or lysosome alteration may be mentioned as oxidative damages of hepatocytes.

The galenical composition of the present invention has an effect to protect hepatocytes from such damages. This effect is considered to be caused by a DPPH radical-scavenging activity of the composition of the present invention.

In the present invention, metals include iron, copper and vanadium.

The galenical composition of the present invention inhibits or prevents hepatocellular carcinogenesis induced by a carcinogen such as diethylnitrosamine (DEN). The galenical composition of the present invention can prevent the early stage of hepatocellular carcinogenesis phenomena, which is showed by an experiment of GST-P that is a stable marker of preneoplastic cells and tumor cells.

Moreover, the galenical composition of the present invention has effects to increase carcinogen-metabolizing enzyme activity. As carcinogen-metabolizing enzymes, a Cytochrome P450 (CYP) isoform, phase I enzyme and phase II enzyme may be mentioned. More specifically, glutathione peroxidase, glutathione reductase, glucose-6-phosphate dehydrogenase, catalase, glutathione-S-transferase and quinone reductase may be mentioned as carcinogen-metabolizing enzymes.

The galenical composition of the present invention has an anticarcinogenic effect of protecting cells and tissues from cytotoxicity/genotoxicity of peroxides and hydroxyl radicals, and modulating the initiation stage of chemical carcinogenesis, by promoting these enzyme activities.

### EXAMPLE 1

In vitro test on the protective effect of YHK against damage by metal ions in hepatocytes and lysosomal fractions

(1) Summary of Experiment

Hepatocytes were isolated from Wistar rats by collagenase perfusion method and cultured as such and also with α-linolenic acid (LNA)-bovine serum albumin (BSA). Hepatocytes were then cultured with graded dilution of YHK (Kyotsujigyo Inc., Tokyo) sample (100 μg/ml and 200 μg/ml) or sylibin (100 μg/ml) dissolved in dimethyl sulfoxide for 10 min before the addition of metallic salts (iron, copper and vanadium). Lysosomal fractions were prepared to carry out lysosome fragility test by measuring β-galactosidase activity and lactate dehydrogenase leakage and oxidative damage tests in the presence of hydrophilic and lipophilic free radical generators.

Digesting activity by DPPH was also assessed. Both YHK and sylibin showed a prominent protective effect against all challenge metal ions, as expressed by the half inhibition concentration (IC₅₀) of against lipid peroxidation and MDA formation. However, YHK seemed to be more effective than sylibin in Fe-induced peroxidative damage (p<0.05). Both test compounds, irrespective of the concentration, significantly reduced the LDH and β-galactosidase concentration in lysosomal fractions. As compared to untreated lysosomal fractions challenged with the two peroxide radical generators, both YHK and sylibin exerted a significant protection. Both compounds showed a comparably prominent DPPH radical-scavenging activity. These data support the potential clinical application of this novel natural product in clinical practice.

(2) Materials and Methods

Isolation and culture of hepatocytes: Male Wistar rats weighing 180 to 210 g were fed with standard chow and water ad libitum. Hepatocytes were isolated by collagenase perfusion method as described by Wolkoff et al. (J Clin Invest 1987; 79: 1259-1268). Briefly, the liver was perfused with collagenase type IV (Sigma Chemical, St. Louis, Mo., USA) and isolated hepatocytes were suspended in culture medium consisting of Waymouth's 752/1 (Gibco, Grand Island, N.Y., USA) containing 5% heat-inactivated fetal bovine serum, 2.5 mM CaCl₂, 5 μg/mL bovine insulin (Sigma), 100 U/mL penicillin and 0.1 mg/mL streptomycin. The isolated cells were further fractionated on Percoll density gradients to obtain a viability higher than 98%, as ascertained by trypan blue. Approximately 1.5×10⁶ cells in 3 mL or approximately 5.0×10⁶ cells in 10 mL in individual 60- or 100-mm diameter Lux culture dishes were placed in an incubator in an atmosphere of 5% CO-95% air at 37° C., and then separately cultured. After a 9 hr incubation, the monolayer of hepatocytes was cultured for an additional 12 hr in the medium containing 1.0 mM α-linolenic acid (LNA)-bovine serum albumin (BSA). Seventy percent or more of added LNA was adsorbed by cultured cells after incubation. The control hepatocytes were maintained in culture in the medium without LNA and the amount of cell protein was measured by the method of Lowry et al. (J Biol Chem 1951; 193: 265-275).

Preparation of YHK sample: YHK was prepared from a hot water extract of 55% by weight of Panax pseudoginseng, 25% by weight of Eucommiae ulmoides, 10% by weight of Polygonati Rhizoma, 5% by weight of Licorice root (Glycyrrhiza glabra), 3% by weight of Panax ginseng (Chinese Ginseng) and 2% by weight of honey.

Hepatocyte culture test: Hepatocytes were washed twice with Hanks' medium and further cultured in 60-mm (1.5×10⁶ cells/dish) with graded dilution of the aforementioned YHK (Panax pseudoginseng, Eucommiae ulmoides, Polygonati rhizoma, Licorice root (Glycyrrhiza glabra), Panax ginseng, Kyotsujigyo Inc., Tokyo) sample (100 μg/ml and 200 μg/ml) or sylibin (100 μg/ml) dissolved in dimethyl sulfoxide for 10 min before the addition of metallic salts dissolved in 100 μM saline. After incubation for 6 hr, the medium was separated. Malonyldialdehyde (MDA) in the medium was assessed by a slight modification of the Uchiyama and Mihara method (Anal Biochem 1978; 86: 271-278). Briefly, to 0.1 ml of the medium in a 12 ml glass tube, 3 ml of 1% phosphatidic acid and 1 ml of 0.67% thiobarbituric acid were added and heated at 100° C. for 45 min. After cooling in ice water, 4 ml of n-butanol was added and the resulting mixture was shaken and then centrifuged to separate the butanol layer. The fluorescence intensity in the butanol layer was assayed at the excitation and emission wavelengths of 515 and 553 nm, respectively. The auto-oxidation products of fatty acid in the medium were within 0.3 nmol and were used as blank. Dimethyl sulfoxide (20 μl) was diluted in 2000 μl of the culture medium, including control cultures of metal ions only in the absence of the test compounds and the final concentration of 1% dimethyl sulfoxide had no measurable effect on lipid peroxidation in basal cultured hepatocytes.

Preparation of LNA-BSA complex: LNA was adsorbed to bovine serum albumin by the method of Sugihara et al. (J Pharmacol Exp Ther 1995; 274: 187-293). One mmol of LNA was dissolved in 10 ml of 0.1N NaOH solution. To this solution, serially added were 240 ml of complete Williams medium E 1 mM BSA which had a fatty acid/albumin molar ratio of 4. The resulting fatty acid-BSA complex was sterilized by filter-passage through a 0.2 μm Millipore™ filter.

Preparation of lysosomal fractions: After homogenizing in 9 volumes of 0.3 M sucrose, the liver was centrifuged at 450×g for 10 min. The supernatants were again centrifuged at 3500×g for 10 min, and the lysosome-containing supernatant was centrifuged at 10000×g for 10 min. The pellets were washed and centrifuged at 10000×g for 10 min, and resuspended in the sucrose buffer to a protein concentration of 15 mg/ml. The resultant lysosome enriched fraction was found to be stable in the homogenization buffer at 4° C. for up to 6 h.

Lysosome fragility test: The fraction was incubated with the test compound and each of metal ions and β-galactosidase activity was measured in accordance with the method of Olsson et al. (Anal Cell Pathol 1990; 2: 179-188), using 4-methylumbelliferyl-β-galactosidase as a substrate. The results were expressed as percentage of total β-galactosidase released. Lactate dehydrogenase leakage was also measured in the culture medium in accordance with the method of Hillaire et al. (Hepatology 1995; 22: 82-87).

Oxidative damage tests of lysosomes: Assays for the release of acid phosphatase and β-N-acetylglucosaminidase from lysosomes were carried out by incubating lysosomal suspensions in the presence of test compounds. The incubation was carried out in the presence of 50 mM 2,2′-azobis(2-amidinopropane)dihydrochloride (AAPH) or 1 mM 2,2′-azobis(2,4-dimethylvaleronitrite (AMVN) which are azo-compounds that generate peroxide radicals after thermal hemolysis in aqueous phase and lipid phase, respectively. The effect of the test compounds on cell damage was calculated as a percentage of control. Further, quenching activity of both YHK and sylibin against 1,1-diphenyl-2-picrylhydrazyl (DPPH) radicals was assessed by spectrophotometry. One ml of test solutions and lysosomal suspensions preloaded with the compounds were incubated with 2 ml ethanol solution of 0.25 mM DPPH radicals and 2 ml 0.1M acetate buffer (pH 5.5) for 45 min at 37° C. and then absorbance was measured at 517 nm. For this experiment, lysosomal suspensions were preincubated in the presence of 1 mM of the test compounds for 30 min and centrifuged at 12000×g for 10 min. Then the pellets were washed in 0.15 M KCl-5 mM Tris buffer (pH 7.4), centrifuged and re-suspended in 0.1M acetate buffer (pH 5.5).

Statistical Analysis

All experiments were repeated three times. Significance was established by analysis of variance and the level of significance was determined by employing a Duncan's multiple-range test. Data were expressed in the text as means (SD) and a probability value of <0.05 was set as indicating that a statistically significant difference existed between experimental groups.

(3) Results

Metal-induced lipid peroxidation: MDA accumulation in the medium showed a direct time-course increase with the incubation time up to 6 hr after the addition of metal catalysts. The amount of MDA concentration in the presence of Fe, Cu and V ions was 2.8, 2.7 and 2.4 nmol/mg protein/6 hr in normal hepatocytes and 8.8, 6.2 and 10.7 nmol/mg protein/6 hr in LNA-loaded hepatocytes, respectively. These data are in agreement with the findings of Furuno et al. (J Toxicol Environm Health 1996; 48: 121-129). Both sylibin and YHK significantly decreased at the same extent MDA generation in the medium (p<0.05). As shown in Tables 1 and 2, both YHK and sylibin showed a potent protective effect against all challenge metal ions, as expressed by the 50% inhibition concentration (IC₅₀) of against lipid peroxidation. Fe-induced lipid peroxidation either in normal hepatocytes and in LNA-loaded hepatocytes was suppressed by both test compounds at a significantly lesser extent than in Cu- and V-induced hepatocytes (p<0.05). Both compounds, irrespective of the concentration, were significantly effective in suppressing Cu- and V-induced lipid peroxidation in normal and LNA-loaded cells at a comparable level. On a molar ratio, the protective effect of YHK against Fe-induced peroxidative damage, either on normal hepatocytes and in LNA-loaded cell, was comparable to sylibin. However, YHK at higher concentration was further more effective (p<0.05). In the case of sylibin, higher concentration did not improve the effect.

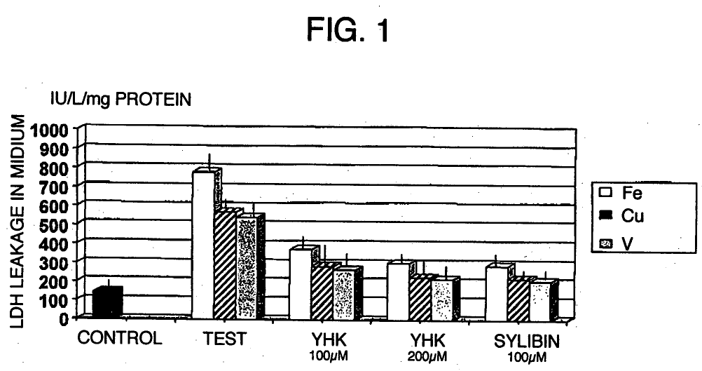

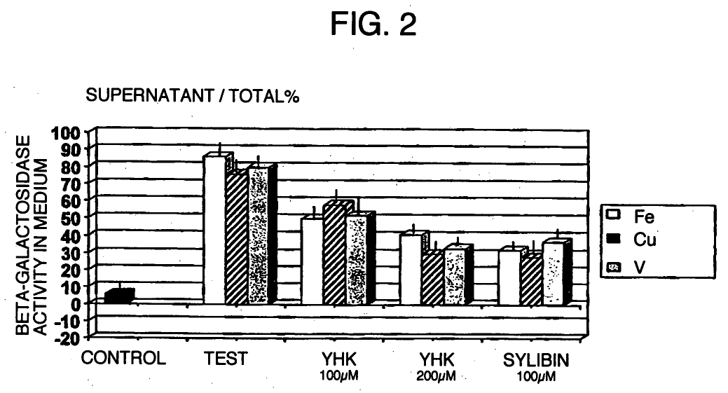

Lysosomal fragility test: In the presence of metal ions, lysosomal fractions expressed a significant increase of LDH leakage and β-galactosidase release (p<0.01), as shown in FIGS. 1 and 2. Both test compounds, irrespective of the concentration, significantly reduced the LDH concentration in the medium of lysosomal fractions (p<0.05). Sylibin and the higher concentration of YHK significantly decreased the β-galactosidase release from lysosomes (p<0.05, FIG. 2).

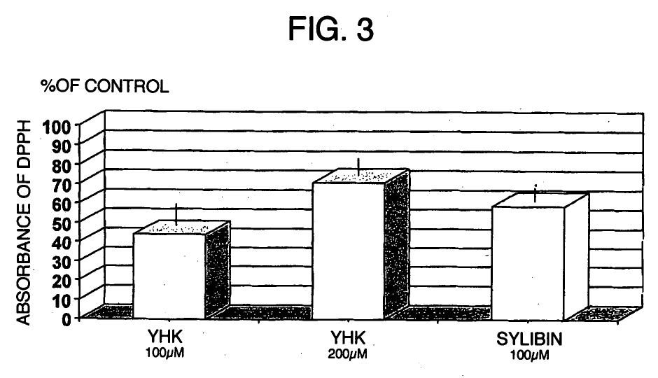

Tests of lysosomal oxidative stress: As compared to untreated lysosomal fractions challenged with the two peroxide radicals generators, both YHK and sylibin exerted a significant protection (p<0.01, Table 3). In particular, such protection was comparably effective between hydrophilic and lipophilic generated free radicals. YHK showed a significantly more protective effect than sylibin against lipophilic generators (p<0.05). Both compounds showed a comparably significant DPPH radical-scavenging activity (p<0.01, FIG. 3).

(4) Discussions

It has been shown that metals, including iron, copper, and vanadium undergo redox cycling, while cadmium, mercury, nickel and lead decreases glutathione and protein-bound sulfhydryl groups, resulting in the production of reactive oxygen species as superoxide ion, hydrogen peroxide, and hydroxyl radical. Indeed, the most important mechanism of oxidative damage to proteins is metal-catalyzed oxidation which may end up in the loss of enzymatic activity and alteration of protein structure. This process involves generation of H₂O₂ and reduction of Fe or Cu by a suitable electron donor like NADH, NADPH, ascorbate and others. Fe and Cu ions bind to specific metal binding sites on proteins and react with H₂O₂ to generate OH and the resulting highly reactive oxygen species attacks amino acid residues. There is evidences suggesting the role of free radical generation and oxidant injury in the pathogenesis of liver injury and fibrosis in metal storage diseases. Although several antioxidants may decrease oxidative stress-related tissue damage, there are concerns over toxicity of some synthetic analogues such as phenolic compounds and to date there are only scanty clinical reports. In the present in vitro study, the galenical composition of the present invention showed to far more significantly protect hepatocytes from metal ions-induced lipid peroxidation at even better extent than sylibin. This is an interesting findings considering that, to the contrary of many herbal remedies experimentally tested, the present phytotherapeutic composition has shown to significantly lower within three weeks the ALT level in the majority of HCV-related chronic liver disease patients (Non-Patent Document 6) and, moreover, to decrease Maruyama score in an awarded pilot clinical study done on the same subjects (Non-Patent Document 7). It has been proved that free radicals-modified membrane lipids and proteins in hepatic iron overload bring about a derangement of hepatic microsomal enzyme activity, electron transport, respiration and lysosomal function. AAPH and AMVN are azo-compounds which generate radicals after thermal homolysis in aqueous phase and lipid phase, respectively, and our findings showed that YHK significantly protects lysosomal integrity with a mitigated LDH and β-galactosidase release. This is likely to be the result of its effective DPPH radical-scavenging activity and its activity against lipophilic-generators of free radicals which was stronger than sylibin. Indeed, during metal-induced injury, the oxidant stress damage is preferentially targeted to the lysosomal fraction which is particularly rich in low molecular weight redox-active iron and the rupture of lysosomes, followed by relocation of labile iron to the nucleus, could be an important intermediary step in the generation of oxidative DNA damage, as it has been very recently demonstrated (Kurz et al. Biochem J. 2003; 11 in press). These latter findings are of interest in view of recent data suggesting that metal-induced lysosome alterations are advocated among the mechanisms of liver carcinogenesis.

TABLE 1
Inhibiting activity of YHK and sylibin on FeSO₄, CuSO₄
and VCl₃-induced lipid peroxidation in normal
hepatocytes (mean ± SD)

| | |
|---|---|
| YHK | Sylibin |

| Metal ion | 100 μM | 200 μM | 100 μM |
|---|---|---|---|
| FeSO₄ | 15.6 ± 4.6^{§} | 12.2 ± 4.4^{§}* | 18.9 ± 3.2^{§} |
| CuSO₄ | 7.9 ± 0.3 | 6.7 ± 0.7 | 7.3 ± 0.3 |
| VCl₃ | 8.7 ± 0.99 | 9.4 ± 0.85 | 10.8 ± 1.2 |

Values represent the concentrations that inhibit lipid peroxidation by 50% (IC₅₀, μM). IC₅₀ was calculated from the concentration-activity curves.
^{§}p < 0.05 vs CuSO₄ and VCl₃.
*p < 0.05 vs Silybin

TABLE 2
Inhibiting activity of YHK and sylibin on FeSO₄, CuSO₄
and VCl₃-induced lipid peroxidation in LNA-loaded cells

| | |
|---|---|
| YHK | Sylibin |

| Metal ion | 100 μM | 200 μM | 100 μM |
|---|---|---|---|
| FeSO₄ | 73.4 ± 7.4^{§} | 59.2 ± 9.2^{§}* | 79.9 ± 9.2^{§} |
| CuSO₄ | 15.9 ± 2.2 | 19.8 ± 1.7 | 16.8 ± 1.5 |
| VCl₃ | 16.7 ± 1.2 | 18.1 ± 0.57 | 17.3 ± 1.2 |

Values represent the concentrations that inhibit lipid peroxidation by 50% (IC₅₀, μM). IC₅₀ was calculated from the concentration-activity curves.
^{§}p < 0.05 vs CuSO₄ and VCl₃.
*p < 0.05 vs silybin

TABLE 3
Effect of YKH (K-17.22) on the release of lysosomal
enzymes in the presence of hydrophilic or lipophilic
radical generators: enzyme activity (% of control ± SE)

| | |
|---|---|
| Acid phosphatase | β-N-acetylglucosaminidase |

AAPH-induced release

| | | |
|---|---|---|
| YHK 10⁻⁴M | 52.4 ± 6.1* | 47.7 ± 4.2* |
| Sylibin 10⁻⁴M | 51.9 ± 5.6* | 54.6 ± 4.7* |

AMVN-induced release

| | | |
|---|---|---|
| YHK 10⁻⁴M | 64.4 ± 7.9*^{§} | 61.3 ± 8.7*^{§} |
| Sylibin 10⁻⁴M | 83.9 ± 10.4* | 77.3 ± 7.4* |

*p < 0.01 vs DMSO (control compound)
^{§}p < 0.05 vs. sylibin

### EXAMPLE 2

Inhibitory Effect of YHK on Early Stage of Liver Neoplastic Lesions

(1) Summary of Experiment

The aim of this study was to investigate the effects of YHK on hepatocarcinogenesis induced by diethylnitrosamine (DEN) in Sprague Dawley rats. Rats were randomly divided into 3 groups and followed up for 15 weeks. Groups 1 was given standard food and represented the healthy control. Liver preneoplastic foci were induced using the DEN method in groups 2 and 3 (20 rats each). However, group 3 was concomitantly given 50 mg/kg/day of YHK. For quantitative assessment of liver preneoplastic foci, the placental form of glutathione-S-transferase (GST-P) positive foci were measured using immunohistochemical staining and image analysis. Treatment using DEN caused a significant decrease in body weight and increase in liver weight compared to the control group while concomitant supplementation with YHK prevented body weight loss and liver weight increase. As compared to DEN-only treated rats, the group given YHK showed a significant decrease in the number, size and volume of GST-P-positive foci. Moreover, co-administration of YHK significantly reduced the incidence, number, size and volume of hepatocellular carcinoma. Anti-inflammatory, anti-fibrotic as well as antioxidative properties of this compound are mechanisms which are likely to be advocated for to exaplain its protective effect. It is concluded that YHK by preventing hepatocarcinogenesis in DEN-induced liver neoplastic lesions in rats has the potential to a large clinical application as a functional food.

(2) Materials and Methods

Sprague Dawley rats were housed and maintained in 12-hour light/dark cycles at 23° with a humidity of 60%/10% in an environmentally-controlled vivarium (temperature, ventilation, humidity and light-dark cycle) and with free access to deionized water and non-nutrient fibers ad libitum. The animals were kept for 15 weeks under such conditions.

Preparation of YHK sample: YHK was prepared in the same manner as in Example 1.

Experimental protocol: Sixty rats were randomly divided into 3 groups of 20 rats each and treated as follows until the end of the experiment: Group 1 was given regular chow pellet as served as healthy control; Group 2, given standard chow pellet and Group 3, given the standard chow pellet containing YHK calculated as to assure a daily intake of 50 mg/kg, represented the hepatocarcinogenesis model. Thus, they received a single intraperitoneal injection of diethylnitrosamine (DEN) (200 mg/kg/bw in saline) in accordance with the method of Solt and Farber (Nature 1976; 263: 701-703) with modification. The proper mixture between standard food and powdered YHK was prepared each day and the food trays were checked every day, cleared of debris, weighed and filled.

Histopathological analysis and glutathione S-transferase placental form (GST-P) staining and counting: At the end of the 15-week study period, rats were sacrificed and macroscopic examination was performed to detect any external pathology. Livers were then excised and weighed. Then, 5 mm-thick slices were cut from each lobe in individual rats and quickly fixed in cold acetone (0 to 4° C.) for 6 h. The slices were then taken out and embedded in paraffin for subsequent immunohistochemical examination of GST-P. GST-P positive foci (defined as lesions of the cells of more than 0.01 mm² in area) were assayed by an immunohistochemical method using a streptavidin-biotin-peroxidase complex (ABC) in accordance with the method of Hsu et al. (J. Histochem. Cytochem 1981; 29: 577-580). Briefly, after being deparaffinized with xylene, quenched with hydrogen peroxide and blocked with normal serum, the liver tissue sections were treated sequentially with normal goat serum, anti-rabbit GST-P antibody (1:2000), biotin-labeled goat anti-rabbit IgG (1:400) and finally with ABC. The diaminobenzidine method was used to demonstrate the sites of peroxidase binding. For quantitative assessment of lesions it was considered: the number of GST-positive foci/cm², the percentage of section area occupied by the foci and diameters of GST-P-positive foci and nodules >0.2 mm, by using an image analyzer in accordance with the method of the following document (Pugh et al. Cancer Res 1983; 43: 1261-1268, Campbell et al. Cancer Res 1982; 42: 465-472). Liver lesions were diagnosed according to the criteria described by Squire and Levitt and the descriptions given following the guidelines of the Institute of Laboratory Animal Resources.

Statistical Analysis

Results are expressed as mean±s.d. Statistical analysis was performed using an SPSS programme for Windows® XP. The differences between groups were evaluated using one way analysis of variance, followed by Dunnette's test for pair-wise comparison and Tukey's family error rate. In all cases, P<0.05 was considered as the minimum level of statistical significance.

(3) Results

Body and Liver Weight

All the rats survived in good condition until the scheduled sacrifices. Treatment with DEN significantly decreased the body weight (p<0.05) and increased the liver weight (p<0.05) compared to the control group (Table 4). Oral intake of YHK was proved to significantly inhibit DEN-induced rat body weight loss and liver weight increase (p<0.05).

TABLE 4
Body and liver weight changes during DEN-
induced hepatocarcinogenesis:
Effect of dietary supplementation of YHK

| | | Body weight | | Liver |
|---|---|---|---|---|
| | | Start of the | End of the | weight |
| Group | Treatment | experiment | experiment | Grams |
| 1 | Standard food | 127 ± 1 | 353 ± 9 | 14.7 ± 0.5 |
| | only | | | |
| 2 | DEN | 125 ± 2 | 321 ± 5* | 16.9 ± 0.5* |
| 3 | DEN + YHK | 126 ± 1 | 349 ± 8§ | 15.0 ± 0.6§ |
| | 50 mg/kg/day | | | |

*p < 0.05 vs YHK-intake rats and vs healthy control rats
§p < 0.05 vs DEN-only treated rats

Assessment of GST-Positive Hepatocellular Foci

The results of quantitative analysis of the frequency of GST-P-positive foci are summarized in Table 5. The two-dimensional assessment showed that GST-P-positive lesions were significantly lesser in rats administered YHK (group 3) than in group 2. The same results appeared when the statistical analysis was applied to volumetric assessment too such as number of lesions per cm³, mean volume and the volume when expressed as a percentage of parenchyma of GST-P-positive lesions.

TABLE 5
Number and size of GST-P-positive hepatic lesions in
DEN-induced hepatocarcinogenesis:
Effect of dietary supplementation of YHK

| | Group | |
|---|---|---|
| | 2 | 3 |
| | DEN | DEN + YHK 50 mg/kg/day |
| No./cm² | 96 ± 4 | 71 ± 4 |
| Mean area (mm²) | 0.32 ± 0.04 | 0.25 ± 0.03* |
| No./cm³ | 2012 ± 133 | 1545 ± 109* |
| Mean volume (mm³) | 0.17 ± 0.03 | 0.14 ± 0.2* |
| Foci/parenchyma % | 28.2 ± 2.5 | 21.7 ± 2.1* |

*p < 0.05 vs DEN-only treated rats

Tumor incidence: No liver tumor was detected in untreated rats while tumor hepatocellular origin was observed in DEN-treated rats (groups 2 and 3) (Table 6). The incidences of tumor of group 3 were significantly lower than those of group 2 (p<0.01). The multiplicities and total number of the tumors for groups 3 were significantly smaller than the corresponding values for group 2 (p<0.05), when also assessed by volumetric calculation.

TABLE 6
Incidence, number, size and volume of DEN-induced
hepatocellular carcinoma:
Effect of concomitant supplementation with YHK

| | Group | |
|---|---|---|
| | 2 | 3 |
| | DEN | DEN + YHK 50 mg/kg/day |
| No. of rats with | 96 ± 4 | 71 ± 4 |
| HCC (%) | | |
| Mean area (mm²) | 1.40 ± 0.47 | 0.17 ± 0.09* |
| No./cm³ | 1.3 ± 0.3 | 0.8 ± 0.2* |
| Mean volume (mm³) | 0.79 ± 0.28 | 0.02 ± 0.01* |
| HCC/parenchyma % | 0.7 ± 0.2 | 0.2 ± 0.1* |

*p < 0.05 vs DEN-only treated rats

(4) Discussions

Hepatocellular carcinoma (HCC) is a devastating and increasingly common disease and progress in the management of this cancer has been slow while a high rate of recurrence is still a limiting factor in the success of surgical resection. While hepatitis C and B, and aflatoxin in some areas, are the main cause of HCC, there is an increasing concern over the wider involvement of xenobiotics in carcinogenesis. Indeed, there are many genotoxic carcinogens occurring naturally in our environment, such as the large group of heterocyclic amine mutagens. A number of chemicals agents are currently employed to experimentally mimick such condition since genotoxic carcinogens can induce irreversible DNA damage in primary cells which then the primary cells proliferate clonally in the presence of promoter substances until they acquire self-sustaining growth capability. Classically, chemical hepatocarcinogenesis is regarded as a multistep process with at least three stages, i.e. initiation, promotion and progression, and each of these steps involves host biochemical, endocrinological, immunological, and microenvironmental regulatory systems. On a practical ground, DNA can be damaged along the whole process of absorption of carcinogens into the body, distribution to most sensitive tissues, metabolism which gives rise to a further form reacting with DNA, detoxification, and excretion. In this instance, a protective dietary approach would represents an ideal strategy when considering that there is an established evidence that diet plays a major role in the prevention of many diseases, including cancer. Thus, nowadays there is an increasing literature supporting the benefit of specific nutrients which, back in the early 1980s, had been termed in Japan as “functional foods”. In the present experiment we employed YHK which has been shown to exert potent hepatoprotective properties in several experimental models of liver injury. However, unlike other natural remedies which are regarded as limited in long-term treatment, this composition can be safely integrated in normal diet and long-term studies have proved to significantly exert a transaminases-lowering effect in HCV-related cirrhotic patients (Buetler et al. Biochem Biophys Res Comm 1992; 188: 597-603; Aceto et al. Carcinogenesis 1990; 11: 2267-2269). This experiment showed that this composition, when orally ingested concomitantly with an established chemical hepatotoxin, could prevent the early phase of carcinogenesis, as expressed by the experiment of GST-P which is a stable marker for persistent preneoplastic and neoplastic cells not only at the protein but also at the mRNA level throughout hepatocarcinogenesis in rats. Overall, the GSTs are a family of dimeric proteins (labelled as Alpha, Mu, Pi, Theta, Sigma, Kappa, and Zeta) that play important roles in both the intracellular transport of hydrophobic molecule and the metabolism of toxic compounds. GST-P protein is hardly detectable in normal rat liver but becomes expressed and detectable in hyperplastic nodules and hepatocellular carcinomas, irrespective of the kind of carcinogen used. GST-positive cells are typically characterized by an elevated DNA replication and the growth of GST-P-positive single cells and GST-P-positive liver foci is believed to be results between such replication and the counterbalance determined by death of cells. However, as stated above, a number of chemicals and/or dietary toxins may act as tumor promoters by triggering a progressive cellular damage. In particular, our study showed that the number, size and volume of either GST-P-positive foci and of overt HCC were significantly reduced by co-administration of YHK, the latter event being at an higher significance level. In general, a number of mechanisms underlie the effects of chemopreventive agents, the suppression of lipid peroxidation or DNA adduct formation and the modulation of phase I or II enzymes being among them. Although the mechanism by which YHK provides significant protection against hepatocarcinogenesis is not clear as yet, taken overall, the insofar demonstrated properties of this compound (i.e. antioxidant, anti-inflammatory and anti-fibrotic) are advocated for to explain its prevention of preneoplastic lesion formation. On the other hand, its safety makes it a potential functional food of large clinical application in the quest to achieve a better control of HCC transformation in chronic liver disease. This holds of particular interest when considering that a number of “would-be” protective natural compound have failed to do the same if not even worsen the carcinogenesis process (Barbisan et al. Cancer Sci 2003; 94: 188-192; Low-Baselli et al. Carcinogenesis 2000; 21: 1869-1877).

### EXAMPLE 3

Beneficial Effect of YHK on Carcinogen-Metabolizing Enzyme Activity in the Liver

(1) Summary of Experiment

In this experiment we investigated the effect of YHK dietary supplementation on the activities of antioxidant, phase I and phase II metabolizing enzymes involved in detoxification as well on liver antioxidant defense system in rats. YHK was administered for four weeks to Wister rats. At the end of the treatment period, different cytochrome P450 (CYP) isoform and phase II enzyme activities were determined by incubation of the liver microsomes or cytosols with appropriate substrates. Dietary supplementation of YHK (2%, w/v) to male rats for four weeks significantly increased the activities of glutathione peroxidase and catalase in liver as compared with corresponding normal diet fed control (P<0.05-0.001). CYP 1A2 activity was markedly increased in all the YHK treatment groups (P<0.05). CYP 1A1 activity was increased significantly in all the groups. Parallel to these changes, YHK feeding to rats also resulted in a considerable enhancement in the activity of phase I and II metabolizing enzymes such as glutathione S-transferase activity to 1.6 fold (and 1.8 fold in liver) as compared with corresponding normal diet fed control (P<0.05-0.01). The induction of such detoxifying enzymes by YHK suggest the potential value of this compound as protective agent against chemical carcinogensis and other forms of electrophilic toxicity. The significance of these results is that YHK has cancer preventive effects against the induction of tumors in various target organs.

(2) Materials and Methods

Preparation of YHK sample: YHK was prepared in the same manner as in Example 1.

Animals were housed in stainless steel wire-mesh cages and kept in an environmentally-controlled vivarium (temperature, ventilation, humidity and light-dark cycle) and with free access to food (commercial rodent diet). The animals were fed for five days to be conditioned before the study.

Experimental protocol: For studying the effect of dietary supplementation of YHK on antioxidant, phase I and phase II metabolizing enzymes, the rats were divided into control and experimental groups consisting of twenty animals in each group. These animals were fed with either normal diet (control group) or 2% YHK diet (experimental group) which was prepared by mixing normal diet and YHK, with a final concentration of YHK fixed at 2%. This defined feeding regimen was kept for four weeks. The selection of dose of YHK was based on previous studies where significant cancer chemopreventive effects were observed when added to either adriamycin and/or cis-platinum. After four weeks, the animals were sacrificed by cervical dislocation, and whole liver was immediately removed, rinsed in an aqueous cold 0.9% sodium chloride solution and then perfused with cold 0.85% sodium chloride and homogenized in chilled 0.1M phosphate buffer (pH 7.4) containing 1.17% potassium chloride using a Potter-type Teflon® glass homogenizer. Parts of the homogenate were centrifuged at 800 g for 15 min at 4° C. using Hitachi cold centrifuge model CR15B to separate nuclear debris. The aliquot so obtained was centrifuged at 12,000 rpm for 30 min at 4° C. to obtain postmitochondrial supernatant which was used as a source of enzymes. The rest of the sample was used for liver microsomes and cytosols extraction which was carried out by a differential centrifugation method. That is, homogenates were subjected to centrifugation for 15 min at 4° C. in a refrigerated centrifuge (OM 3593 IEC Co. Ltd. USA). The supernatant was centrifuged at 105 000×g for 60 min at 4° C. in a preparative ultracentrifuge (20PR-52D; Hitachi, Tokyo). The pellet of microsomes was suspended in the homogenization solution in the homogenizer and centrifuged again. The supernatant (cytosol fraction) after discarding any floating lipid layer and appropriate dilution, was used for enzyme assays in accordance with the method described above (Robson et al, Br J Clin Pharmacol 1987; 24: 293-300), and the remaining supernatant was stored in 20 mM phosphate buffer (pH 7.4) containing 20% wv glycerol at −80° C. until analysis. The microsomal protein content was determined by the method of Lowry et al. (J Biol Chem 1951; 193: 265-275). The P450 content was determined by the method of Omura & Sato (J. Biol. Chem. 1964; 239: 2370-2379).

Liver Antioxidant Assay

Glutathione peroxidase activity was measured in accordance with the method of Mohandas et al. (Cancer Res. 1984; 44: 5086-5091). The reaction mixture was prepared with 1.44 ml of 0.1M phosphate buffer (pH 7.4), 0.1 ml of 0.5 mM EDTA, 0.1 ml of 1.0 mM sodium azide, 0.05 ml of glutathione reductase (1.0 EU/ml), 0.1 ml of 1.0 mM GSH, 0.1 ml of 0.1 mM NADPH, 0.1 ml of 0.019M hydrogen peroxide, 0.025 ml of renal PMS (10% w/v) and 0.05 ml of hepatic PMS (10% w/v) in a total volume of 2.0 ml. Enzyme activity was calculated as nmol NADPH oxidized/min/mg protein using a molar extinction coefficient of 6.22×10³ M/cm. Catalase activity was determined by the method of Claiborne (Claiborne, A.: Catalase activity. In: CRC Hand Book of Methods for Oxygen Radical Research. Ed.: R. A. Green Wald. CRC Press, Boca Raton, Fla., 1985 pp. 283-284) and then modified in accordance with the method of Ansar et al. (1999). That is, the assay mixture was produced with 1.0 ml of 0.05M phosphate buffer (pH 7.0), 0.975 ml of 0.019M hydrogen peroxide, 0.025 ml of renal and hepatic PMS (10% w/v). Catalase activity was calculated by the decomposition rate of hydrogen peroxide measured as a decrease in absorbance at 240 nm.

Cytosol Phase I and Phase II Enzymes

Determination of CYP 1A1/CYP 1A2 activity. The activity of CYP 1A1 and CYP 1A2 was determined using phenacetin as a specific substrate probe in accordance with the method of Tassaneeyakul et al (J. Pharmacol. Exp. Ther. 1993; 265: 401-407). The activity of the high affinity component (CYP 1A2) of phenacetin-O-deethylase was determined by incubating 5 ml of phenacetin with liver microsomes (0.5 mg mL⁻¹) for 30 min. The reaction was terminated by addition of 1M sodium hydroxide. The formation of the metabolite, paracetamol was measured by a specific HPLC method (Tassaneeyakul et al., above). The activity of the low affinity isozyme CYP 1A1 was determined by using phenacetin at a concentration of 300 lm, approximately the Michaelis constant Km of CYP 1A1 reported in rat liver microsomes (Boobis et al 1981). The procedures for incubation and HPLC assay were the same for CYP 1A2. Hepatic cytosolic glutathione-S-transferase activity was determined using a spectrophotometric (340 nm) method (Habig et al. J. Biol. Chem. 1974; 249: 7130-7139) and then modified in accordance with the method of Iqbal et al. (Redox Report, 1996; 2: 385-391). This procedure was based on the enzyme catalysed condensation of glutathione with the model substrate 1-chloro-2,4-dinitrobenzene (CDNB). That is, the reaction mixture consisted of 1.825 ml of 0.1M phosphate buffer (pH 6.5), 0.1 ml of 1.0 mM reduced glutathione, 0.05 ml of 1.0 mM CDNB, 0.025 ml of renal PMS (10% w/v) and 0.01 ml of hepatic PMS (10% w/v), in a total volume of 2.0 ml. The changes in absorbance were recorded at 340 nm and enzyme activity was calculated as nmol formed CDNB conjugate/min/mg protein using a molar extinction coefficient of 9.6×10³M/cm.

Statistical Analysis

Significance was established by analysis of variance and the level of significance was determined by employing a Duncan's multiple-range test. Data were expressed as means (SD) and a probability value of <0.05 was regarded as indicating that a significant difference existed between experimental groups.

(3) Results

The dose of YHK used in the present study did not produce any apparent sign of toxicity such as weight loss or reduced diet and water consumption, throughout the control feeding (data not shown).

The effect of dietary supplementation of YHK to rats on the activities of antioxidant enzymes in liver tissues was evaluated and results are shown in Table 7. Addition of 2% YHK to the diets of 4 weeks old rats for 30 days resulted in a normal weight gain and was well tolerated. The dietary supplementation of YHK resulted in a significant elevations in the activities of glutathione peroxidase and catalase to 118% and 87% as compared with normal diet fed control (P<0.05-0.001). The increase occurred in glutathione peroxidase activity was higher than what observed in catalase activity (Table 8).

The effect of dietary supplementation of YHK on phase II metabolizing enzymes such as glutathione S-transferase is shown in Table 9. The dietary supplementation of YHK enhances the activities of glutathione-S-transferase to about 1.6 fold as compared to animals fed with normal diet (p<0.05-0.001).

Glutathione-S-Transferase Activity

A significant increase in the activity of cytosolic glutathione-S-transferase was observed in the rats treated with YHK (p<0.05). In YHK-fed group, the contents of P450 were significantly increased in male rats (2.66±0.55 nmol.mg MS pro⁻¹) compared with those in the control group which were almost zero (1.08±1.04 nmol.mg MS pro⁻¹). The difference between them were significant (P<0.01). In particular, the CYP 1A was significantly increased by YHK treatment (p<0.05). The results indicated that there was a difference of hepatic microsomal drug-metabolizing enzymes under normal conditions in different sex rats. However, the effect of YHK was comparably effective in either sex (Table 7).

(4) Discussions

Possible mechanism of protection against chemical carcinogensis in which dietary antioxidants protect laboratory animals against the induction of tumors by a variety of chemical carcinogens could be mediated via-antioxidant dependent induction of detoxifying enzymes. More evidences are presented that nutrition plays an important causative role in the initiation, promotion and progression stages of several types of human cancer. The food may contain many chemicals that can antagonize the effects of chemical carcinogens. One of the mechanisms could be by modulation of the enzyme systems involved in the activation and deactivation of chemical carcinogens. A large number of the genotoxic environmental chemicals and natural products, to which man is exposed, require metabolic activation to exhibit their mutagenic and carcinogenic effects. This bioactivation is mainly carried out by some of the phase I enzymes including cytochrome P450 (CYP), which give rise to reactive intermediates that attack DNA and other cellular macromolecules (Smith et al. Annu. N.Y. Acad. Sci. 768: 82±90). Inhibition of bioactivating enzymes and/or induction of detoxication enzymes by either naturally occurring substances or synthetic agents will continue to be a promising chemopreventive strategy.

Cancer prevention may occur by various different mechanisms. These include reduced metabolic toxification and/or enhanced detoxification, which lower the amount of the ultimate initiating carcinogen. Furthermore, in the post-initiation phase, reduced growth of initiated/preneoplastic cells may inhibit the process of tumor promotion.

The activities of hepatic drug-metabolizing enzymes, especially cytochrome P450 and sulfotransferase, were regulated through the sex-related secretion pattern of growth hormone. Some studies reported the sex-related effect on drug-metabolizing enzymes (Kobayashi et al. J Toxicol Sci 2000; 25: 213-222). However, in our study, no marked sex difference in the effects of long-term treatment with YHK on hepatic drug-metabolizing enzymes in rats was observed.

ROS are widely generated in biological system either by normal metabolic pathways or as a consequence of exposure to chemical carcinogen. ROS, by extensive study, may cause membrane dysfunction, protein inactivation, DNA damage and ultimately lead to the multisteps process of carcinogenesis (Sun, Free Radical Bio. Med. 1990, 8, 583-599; Perchellet & Perchellet, Free Radical Biol. Med. 1989, 7, 377-408). The collective action of both antioxidants and phase II enzymes such as glutathione S-transferase and quinone reductase, besides small nonenzymatic water soluble biomolecules, is to afford protection against the adverse effects of oxidants or reactive metabolites of precarcinogens (Sun above; Perchellet & Perchellet, above). Reiners et al. (Carcinogenesis, 1991, 12, 2337-2343), have shown the depleted levels of antioxidant enzymes in 7,12-dimethylbenz(a)anthracene-12-O-tetradecanoylphorbol-13-acetate-treated skin and in skin tumors induced chemically. Depletion of these enzymes following exposure to carcinogens and/or tumor promoter is also known (Sun, above; Perchellet & Perchellet, above). On the contrary, cancer chemoprevention studies have shown that following the administration of chemopreventive agents, the levels of antioxidant enzymes are elevated in various organs of test animals (Wattenberg, Carcinogenasis, 1990, 12, 115-117).

The significant enhancement in the activity of antioxidant enzymes such as glutathione peroxidase, glutathione reductase, glucose-6-phosphate dehydrogenase, catalase and phase II enzymes like glutathione S-transferase and quinone reductase in the various organs of rats fed with YHK suggest that it may contribute to the cancer chemopreventive effects observed with curcumin. These results showed that YHK feeding to rats resulted in the induction of glutathione linked enzymes (which are known to be involved in detoxification of electrophilic product of lipid peroxidation that may contribute to its anti-inflammatory and anti-cancer activities).

The primary antioxidant enzyme catalase possess a low catalytic activity at low intracellular levels of its substrates H₂O₂, under this condition, glutathione peroxidase plays the predominant role in the detoxification of peroxides from the cells and/or tissues (Raes et al. Free Radical Biol. Med. 1987, 3, 3-7). Several reports suggest the pronounced effects of peroxides as compared to O₂ in producing cytotoxicity/genotoxicity in the cellular systems (Sun, above; Perchellet & Perchellet, above). Besides, the highly reactive OH, generated from hydrogen peroxide via the Haber-Weiss-like-Fenton reaction (Perchellet & Perchellet, above), is known to damage macromolecules, specifically DNA, to produce pathological alterations (Sun, above; Perchellet & Perchellet, above). In view of these facts, the enhancement in the activity of glutathione peroxidase and catalase in the liver of YHK-fed rats suggests that such a treatment could protect the cells/tissues against the cytotoxic/genotoxic effects of peroxides and OH.

The two-electron reduction of the metabolic products of polycyclic aromatic hydrocarbons such as quinones, catalyzed by quinone reductase also known as DT-diaphorase, has been considered to be a detoxification pathway, since the resulting hydroquinones may be conjugated and excreted through mercapturic acid pathways. These quinones in addition to electrophilic characteristics, are well known oxidants covalently bind to DNA forming depurinating adducts and play a definitive role in cancer induction (Cavalieri et al. Proc. Natl. Acad. Sci. USA. 1997, 94, 10937-10942). The semiquinone, the product of one electron reduction of quinines via microsomal NADPH-cytochrome P-450, may be toxic or react with molecular oxygen, forming O₂ and regenerating the parent quinines, which is then available for rereduction and thereby undergoes a futile redox cycling. The net result of such a redox cycling is an oxidative stress resulting from disproportionate consumption of cellular reducing equivalent and generation of reactive oxygen species such as O₂, H₂O₂ and OH (Sun, above; Perchellet & Perchellet, above). A phase II enzyme such as glutathione S-transferase not only catalyzes the conjugation of both hydroquinones and epoxides of polycyclic aromatic hydrocarbon with reduced glutathione for their excretion, but also shows low activity towards organic hydroperoxides for their detoxification from cells/tissues (Ketterer, B., K. H. Tan, D. J. Meyer & B. Coles: Glutathione transferases a possible role in the detoxification of DNA and lipid hydroperoxides. In: T. J. Mantle, C. B. Pickett, & J. D. Hayes (eds.), Glutathione S-Transferase and Carcinogenesis, pp. 149-163. New York: Taylor and Francis, 1987). It is therefore reasonable to assume that increased activities of glutathione-S-transferase and quinone reductase in liver of YHK-fed rats play an important role in relation to the cancer chemopreventive effects of this composition.

In conclusion, YHK has anticarcinogenic effect to modulate the initiation stage of chemical carcinogenesis by affecting the enzyme systems that catalyse the activation and detoxification processes. It could be envisaged that the mutagenic and carcinogenic process, and the ultimate risk of developing a chemically-induced cancer, lies in the delicate balance between phase I carcinogen activating enzymes and phase II detoxifying enzymes.

TABLE 7
Effects of long-term YHK consumption on microsomal enzymes

| | Group | P450 nmol/mg MS pro |
|---|---|---|
| ♂ | YHK (n = 10) | 2.66 ± 0.55^{M} |
| | Control (n = 10) | 1.08 ± 1.04 |
| ♀ | YHK (n = 10) | 0.66 ± 0.42 |
| | Control (n = 10) | 0.36 ± 0.18 |

P < 0.01 vs ♂control, P < 0.05 vs ♀control, P < 0.05 vs ♀control.

TABLE 8
Effect of 2% dietary supplementation of YHK to rats on
antioxidant enzyme activities in liver

| Enzyme Activities | Treatment Groups | Liver |
|---|---|---|
| Glutathione peroxidase | Control group | 203.4 ± 9.4 |
| activity | Experimental group | 387.1 ± 7.2^{A} |
| (nmol NADPH | % of control | 173 |
| oxidized/min/mg protein) | | |
| Catalase activity | Control group | 419.9 ± 82.3 |
| (nmol H₂O₂ consumed/min/mg | Experimental group | 658.4 ± 56.3^{B} |
| protein) | % of control | 164 |

Data represent mean ± S.E. of twenty animals. For statistical significance, student's t-test was used between normal diet-fed control and YHK-fed groups.
^{A}p < 0.001.
^{B}p < 0.05.

TABLE 9
Effect of 2% dietary supplementation of YHK to rats on
phase II enzyme activities in the liver

| Enzyme Activities | Treatment Groups | Liver |
|---|---|---|
| Glutathione S- | Control group | 1121.6 ± 119.0 |
| transferase activity | Experimental group | 1692.4 ± 78.2 |
| (nmol CDNB | % of control | 162 |
| oxidized/min/mg | | |
| protein) | | |

Data represent mean±S.E. of twenty animals. For statistical significance, student's t-test was used between normal diet-fed control and YHK diet-fed groups.

What is claimed is:

1. A galenical composition comprising Panax pseudoginseng, Eucommiae ulmoides and Polygonati Rhizoma as essential components, for preventing metal-induced oxidative damage of hepatocytes, for inhibiting hepatocellular carcinogenesis, or for increasing carcinogen-metabolizing enzyme activity.

2. The galenical composition according to claim 1, further comprising at least one selected from the group consisting of Licorice root, Panax ginseng and honey.

3. The galenical composition according to claim 1, further comprising Licorice root and Panax ginseng.

4. The galenical composition according to any one of claims 1 to 3, wherein the composition is for preventing metal-induced oxidative damage of hepatocytes, and the metal is at least one selected from the group consisting of iron, copper and vanadium.

5. The galenical composition according to claim 4, wherein the metal is iron.

6. The galenical composition according to claim 4 or 5, wherein the metal-induced oxidative damage of hepatocytes is lipid peroxidation and/or lysosome alteration.

7. The galenical composition according to any one of claims 1 to 3, wherein the composition is for inhibiting hepatocellular carcinogenesis.

8. The galenical composition according to any one of claims 1 to 3, wherein the composition is for increasing carcinogen-metabolizing enzyme activity.

9. The galenical composition according to claim 8, wherein the carcinogen-metabolizing enzyme is a cytochrome P450 (CYP) isoform, phase I enzyme and phase II enzyme.

10. The galenical composition according to claim 8, wherein the carcinogen-metabolizing enzyme is glutathione peroxidase, glutathione reductase, glucose-6-phosphate dehydrogenase, catalase, glutathione S-transferase or quinone reductase.

11. A method for preventing metal-induced oxidative damage of hepatocytes, for inhibiting hepatocellular carcinogenesis, or for increasing carcinogen-metabolizing enzyme activity, which comprises administering to a subject an effective amount of an extract of a galenical composition comprising Panax pseudoginseng, Eucommiae ulmoides and Polygonati Rhizoma as essential components.

12. The method according to claim 11, wherein the galenical composition further comprises at least one selected from the group consisting of Licorice root, Panax ginseng and honey.

13. The method according to claim 11, wherein the galenical composition further comprises Licorice root and Panax ginseng.

14. The method according to any one of claims 11 to 13, wherein the method is for preventing metal-induced oxidative damage of hepatocytes, and the metal is at least one selected from the group consisting of iron, copper and vanadium.

15. The method according to claim 14, wherein the metal is iron.

16. The method according to claim 14 or 15, wherein the metal-induced oxidative damage of hepatocytes is lipid peroxidation and/or lysosome alteration.

17. The method according to any one of claims 11 to 13, wherein the method is for inhibiting hepatocellular carcinogenesis.

18. The method according to any one of claims 11 to 13, wherein the method is for increasing carcinogen-metabolizing enzyme activity.

19. The method according to claim 18, wherein the carcinogen-metabolizing enzyme is a cytochrome P450 (CYP) isoform, phase I enzyme and phase II enzyme.

20. The method according to claim 18, wherein the carcinogen-metabolizing enzyme is glutathione peroxidase, glutathione reductase, glucose-6-phosphate dehydrogenase, catalase, glutathione S-transferase or quinone reductase.
